(19) United States
(12) Patent Application Publication
(10) Pub. No.: US 2026/0114827 A1
(43) Pub. Date: Apr. 30, 2026

(54) ARM SUPPORT AND METHODS OF USE FOR FEVAR SURGERY

(71) Applicant: Carilion Clinic, Roanoke, VA (US)

(72) Inventors: Robert Fippinger, Roanoke, VA (US); Marilyn Edwards, Roanoke, VA (US); Braiden Saylor, Roanoke, VA (US); Joshua Adams, Roanoke, VA (US); David Ryan King, Roanoke, VA (US)

(21) Appl. No.: 19/371,362

(22) Filed: Oct. 28, 2025

Related U.S. Application Data
(60) Provisional application No. 63/713,312, filed on Oct. 29, 2024.

(51) Int. Cl. A61B 6/00; A61B 6/04; A61G 13/12

(52) U.S. Cl. CPC A61B 6/4441; A61B 6/0407; A61G 13/1235

(57) ABSTRACT

An arm support is provided that allows for convenient positioning of a patient during certain surgical procedures, e.g., FEVAR procedures.

### BACKGROUND

Patients undergoing complex endovascular aortic surgery or fenestrated endovascular aortic repair (FEVAR) surgery are routinely subject to increased radiation exposure. Some studies have shown that elevation of a patient's arms above their head during endovascular surgery may improve visualization by removing the arms from the field of view, and thereby potentially reduce the dose of radiation required. See Pujari et al., “Overhead arm support reduces radiation exposure during complex endovascular aortic repair,” Journal of Vascular Surgery, Vol. 77, Issue 4, April 2023, pp. 991-996.

Current systems for arm support have several drawbacks including the positioning of arms above the patient's head in such a way that compromises the brachial plexus and may result in frozen shoulder syndrome, the inability to positions patient's arms independent of one another, the positioning of patient's arms in a way that obstruct the anesthesiologist's line of sight down the patient's center line from above the head and the positioning of a patient's arms in a way that limits maneuverability of life sustaining devices such as intubation tubes on the patient's center line.

There is a need for an arm support device that can correctly position one or more of a patient's arms while conducting FEVAR surgery, and similar operations, that allows improved access for surgery and lower radiation doses, as well as providing access that is compatible with CT equipment including the positioning of a C-arm relative to the patient on the surgery table.

### SUMMARY OF THE INVENTION

In one aspect, the present invention relates to an arm support comprising: an arm support platform; a base support, the base support comprising a heigh adjustment barrel and a base support plate; a support shaft, the support shaft having an upper portion, a middle portion, and a lower portion, the upper portion being attached to the arm support platform, the middle portion providing a lateral offset that is configured to position the arm support platform over the base support plate, and the lower portion engaging with the height adjustment barrel, wherein the lower portion of the support shaft engages with the height adjustment barrel by sliding up and down within the height adjustment barrel in order to adjust the vertical distance between the arm support platform and the base support plate.

The arm support may be configured to be placed on a surgery table with the base support under the surgery table padding. In one aspect, when the support shaft is at its lowest position within the height adjustment barrel, no portion of the support shaft extends below a lower edge of the surgery table. In another aspect, when the support shaft is at its lowest position within the height adjustment barrel, no portion of the support shaft extends below the lower edge of the height adjustment barrel.

The support shaft may be configured such that the middle portion and the lower portion of the support shaft are joined at 90 degree angle. The support shaft may be configured such that the upper portion and the middle portion of the support shaft are joined at 90 degree angle. The upper portion of the support shaft and the arm support platform may be joined with a tilt adjust mechanism. The height adjustment barrel may comprises a height adjustment clamp that when actuated prevents sliding of the lower portion of the support shaft within the height adjustment barrel.

In one aspect, the upper portion of the support shaft is rotated negative 20 to 70 degrees, preferably negative 30 to 60 degrees, more preferably negative 40 to 50 degrees, and most preferably about negative 45 degrees relative to vertical when the device is configured to support the patient's right arm. Whereas the upper portion of the support shaft is rotated 20 to 70 degrees, negative 30 to 60 degrees, more preferably 40 to 50 degrees, and most preferably about 45 degrees relative to vertical when the device is configured to support the patient's left arm.

In another aspect, the base support plate is transparent to X-ray radiation. The arm support may preferably allow complete freedom of movement of a C-arm fluoroscopy machine for image guided surgery.

The invention also pertains to methods such as methods of positioning a patient on a surgery table comprising elevating one of the patient's arm using a first arm support of claim 1. The methods may also comprise in addition elevating the patient's other arm using a second arm support that is identical to the first arm support.

### BRIEF DESCRIPTION OF THE DRAWINGS

FIG. 1 shows a side view of the arm support mounted on surgical bed under the bed padding with a patient according to an embodiment of the invention;

FIG. 2 shows a front view of the arm support mounted on surgical bed under the bed padding with a patient according to an embodiment of the invention;

FIG. 3 shows a side view of the arm support with the padding removed according to an embodiment of the invention;

FIG. 4 shows the height adjustment barrel with arm support structure removed on a surgical bed with the bed padding removed according to an embodiment of the invention;

### DETAILED DESCRIPTION

The present inventors have designed an improved arm support that provides particular advantages when performing complex aortic surgeries, such as or fenestrated endovascular aortic repair (FEVAR) surgery. The device preferably includes X-ray translucent materials, and can be operated in manner that does not impede the use of C-arm fluoroscopy with the table in order to provide CT imaging of the patient during and or before or after surgery. The arm support also preferably can be used with existing surgery tables, and includes a support plate that slides under the padding of the surgery table. Moreover when the patient is laying on the pad of the surgery table, the patient's weight from the shoulder and back area fully stabilize the lower support plate of the arm support and prevent any unwanted movement of the arm support during surgery.

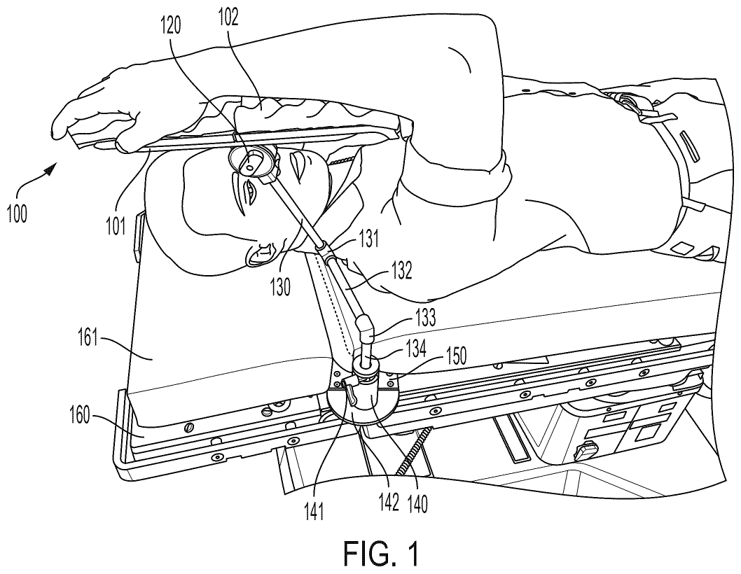

FIG. 1 shows a side view of the arm support 100 mounted on surgical bed 160 under the bed padding 161 with a patient according to an embodiment of the invention. The arm support can move in five degrees of freedom. The arm support includes an arm support platform 101, which optionally includes platform padding 102. The platform 101 can be adjusted with a tilt adjustment knob 120, and is supported with a support shaft that includes a top support shaft 130, a middle support shaft 132, and a lower support shaft 134. The top support shaft is joined to the middle support shaft with an upper bend joint 131. The middle support shaft is joined to the lower support shaft with a lower bend joint. Each bend joint is approximately a 90 degree bend.

The lower support shaft 134 engages with a height adjustment barrel 140. The lower support shaft 134 is configured to slide up and down within the height adjustment barrel 140 in order to adjust the vertical distance between the arm support platform 101 and the lower support platform 150. The height adjustment barrel 140 may include a height adjustment clamp 141 for locking the lower support shaft 134 in place when the desired height is achieved. The lower support platform 150 may be slid under the surgical table padding 160 so that it rests upon the surgical table 160. The height adjustment barrel 140 allows for adjustment of height of the support arm without the need for any portion of the support arm or the support shaft from extending below the bottom edge of the surgical table in a way that might impede the free movement of a C-arm used to produce a CT scan of the patient.

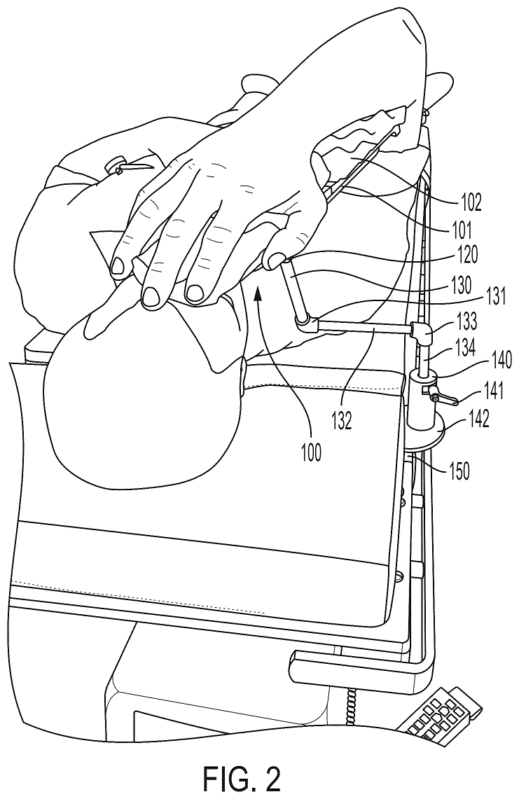

FIG. 2 shows another view of the arm support showing further details on how the upper support shaft 130 is positioned at an angle relative to the middle support shaft 132. The upper portion of the support shaft 130 is rotated negative 20 to 70 degrees, preferably negative 30 to 60 degrees, more preferably negative 40 to 50 degrees, and most preferably about negative 45 degrees relative to vertical when the device is configured to support the patient's right arm. The opposite or non-negative angles may be used when the device is placed on the other side of the table to support the patient's left arm. The lower bend joint and/or upper bend joint may include alternative positions that allow supporting the angles above and switching them when the device is to be placed on the other side of the patient.

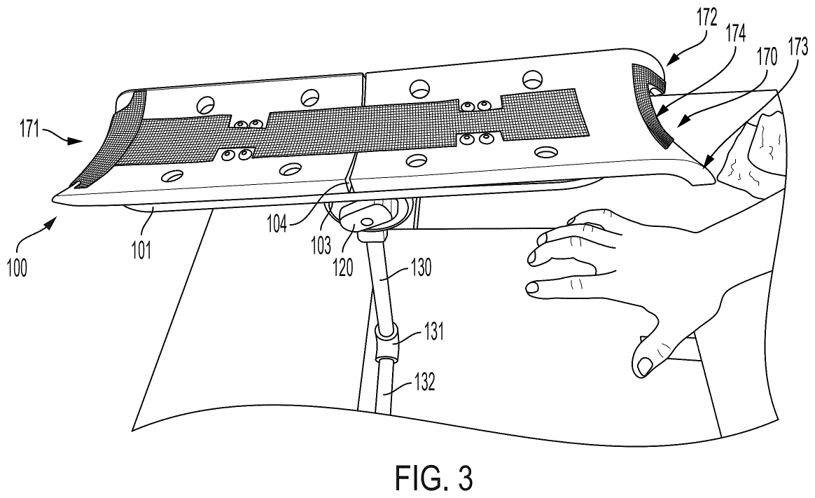

FIG. 3 shows another view where the arm support platform 101 is not obstructed by the patient's arm or the platform padding 102. The support plate is shown with curvature at either end 170 or 171. The ends may include a cut out 172 in the middle that allows good blood flow for the patient and also provides clearance for catheters or blood draw lines that are connected to the patient.

The arm support has a first distal end 170 and a second distal end 171. In one aspect, the first distal end 170 may include a cutout 174 and side portions 172, 173. In another aspect, the side portions 172, 173 may have a downward sloping contour as shown in FIG. 3. The provision of the cutout 174 allows a patient's elbow to more comfortably bend near the first distal end 170 of the arm support platform 101. This can be particularly helpful when the patent's arm has catheters, cannulas or tubes (e.g., intravenous tubes) attached thereto. In this case, the cutout 174 provides additional clearance for such items. The cutout 174 can optionally be included on both the first distal end 170 and the second distal end 171 so that the orientation of the arm support platform 101 may be reversed (e.g., switched to the other side of the patient) without a need to rotate the direction of the arm support platform 101.

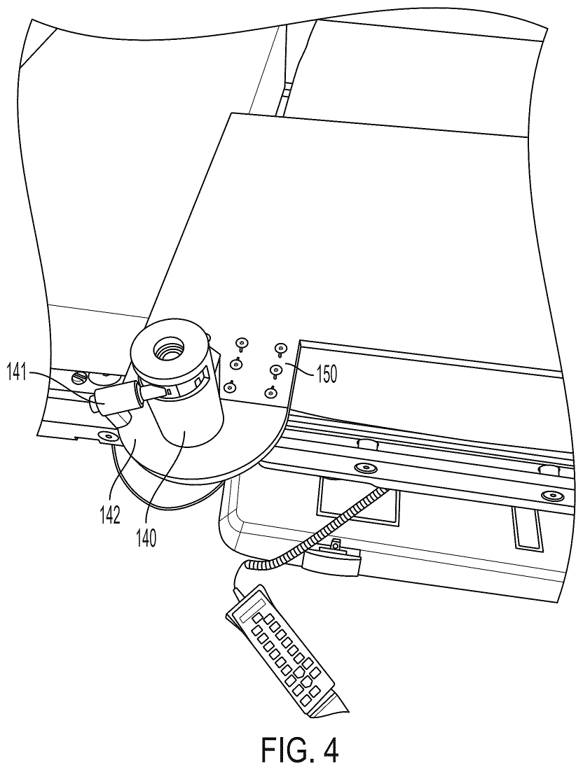

FIG. 4 shows the height adjustment barrel 140 where the lower support shaft 134 (as well as the remaining arm support assembly) is removed. The heigh adjustment clamp 141 is shown as well as the barrel base support platform 142 that allows the barrel base to extend to the patient's side. Also shown is the lower support platform 150 without the surgery table padding 161 in place.

Other embodiments and uses of the invention will be apparent to those skilled in the art from consideration of the specification and practice of the invention disclosed herein. All references cited herein, including all U.S. and foreign patents and patent applications, are specifically and entirely hereby incorporated herein by reference. It is intended that the specification and examples be considered exemplary only, with the true scope and spirit of the invention indicated by the following claims.

What is claimed is:

1. An arm support comprising:
an arm support platform;
a base support, the base support comprising a heigh adjustment barrel and a base support plate;
a support shaft, the support shaft having an upper portion, a middle portion, and a lower portion, the upper portion being attached to the arm support platform, the middle portion providing a lateral offset that is configured to position the arm support platform over the base support plate, and the lower portion engaging with the height adjustment barrel,
wherein the lower portion of the support shaft engages with the height adjustment barrel by sliding up and down within the height adjustment barrel in order to adjust the vertical distance between the arm support platform and the base support plate.

2. The arm support of claim 1, wherein the arm support is configured to be placed on a surgery table with the base support under the surgery table padding.

3. The arm support of claim 2, wherein when the support shaft is at its lowest position within the height adjustment barrel, no portion of the support shaft extends below a lower edge of the surgery table.

4. The arm support of claim 2, wherein when the support shaft is at its lowest position within the height adjustment barrel, no portion of the support shaft extends below the lower edge of the height adjustment barrel.

5. The arm support of claim 1, wherein the middle portion and the lower portion of the support shaft are joined at 90 degree angle.

6. The arm support of claim 1, wherein the upper portion and the middle portion of the support shaft are joined at 90 degree angle.

7. The arm support of claim 1, wherein the upper portion of the support shaft and the arm support platform are joined with a tilt adjust mechanism.

8. The arm support of claim 1, wherein the height adjustment barrel comprises a height adjustment clamp that when actuated prevents sliding of the lower portion of the support shaft within the height adjustment barrel.

9. The arm support of claim 1, wherein the upper portion of the support shaft is rotated negative 20 to 70 degrees, preferably negative 30 to 60 degrees, more preferably negative 40 to 50 degrees, and most preferably about negative 45 degrees relative to vertical when the device is configured to support the patient's right arm.

10. The arm support of claim 1, wherein the upper portion of the support shaft is rotated 20 to 70 degrees, negative 30 to 60 degrees, more preferably 40 to 50 degrees, and most preferably about 45 degrees relative to vertical when the device is configured to support the patient's left arm.

11. The arm support of claim 1, wherein at least the base support plate is transparent to X-ray radiation.

12. The arm support of claim 1, wherein the arm support allows complete freedom of movement of a C-arm fluoroscopy machine for image guided surgery.

13. A method of positioning a patient on a surgery table comprising elevating one of the patient's arm using a first arm support of claim 1.

14. The method of positioning a patient on a surgery table of claim 13, further comprising elevating the patient's other arm using a second arm support that is identical to the first arm support.

15. The arm support of claim 1, wherein at least the arm support platform is transparent to X-ray radiation.

16. The arm support of claim 1, wherein at least the arm support platform and the base support plate is transparent to X-ray radiation.

17. The arm support of claim 1, wherein the arm support platform comprise at least one cutout at a distal end of the arm support platform.

18. The arm support of claim 17, wherein the arm support platform comprises at least side portions on both sides of the cutout portion.

19. The arm support of claim 18, wherein the side portions include a curved surface.

20. The arm support of claim 17, wherein the cutout is adapted to provide clearance for one or more IV tubes, catheters or cannulas attached to a patient for which the arm support is being applied.
